SEQUENCE LISTING (22 sequences)

SEQ ID NO: 1 - DNA, 26 bp, Artificial Sequence
  Primer for FMR1
  misc_feature (9)..(15): Wherein D = A, G or T
agcgatgcdddddddgcatcgcttga

SEQ ID NO: 2 - DNA, 9 bp, Homo sapiens
gtgcacctc

SEQ ID NO: 3 - DNA, 145 bp, Homo sapiens
cggtgataggttgtattgtttcgcgagggttagaatgtttatttttgtagaggtgtatttatgtgatgttatatgtgtattgtatgagtgtatttttgtagaaatgggcgttttggttttcgcgaggtagtgtgatttgttatcg

SEQ ID NO: 4 - DNA, 145 bp, Homo sapiens
cggtgataggtcgtattgtttcgtgagggttagaatgtttatttttgtagaggtgtatttatgtgatgtggtttgtgtattgtatgagtgtatttttgtagaaatgggcgttttggtttttgcgaggtagtgcgatttgttatcg

SEQ ID NO: 5 - DNA, 145 bp, Homo sapiens; identical to SEQ ID NO: 4

SEQ ID NO: 6 - DNA, 145 bp, Homo sapiens
tggtgataggttgtattgttttgtgagggttagaatgtttatttttgtagaggtgtatttaagtgatgttgtgatggtattgtttgagtgtatttttgtagaaatgggtgttttggtttttgtgaggtagtgtgatttgttattg

SEQ ID NO: 7 - DNA, 145 bp, Homo sapiens
tggtgataggttgtattgttttgtgagggttagaatgtttatttttgtagaggtgtatttaagtgatgtaggtgatgtattgtttgagtgtatttttgtagaaatgggtgttttggtttttgtgaggtagtgtgatttgttattg

SEQ ID NO: 8 - DNA, 145 bp, Homo sapiens; identical to SEQ ID NO: 7

SEQ ID NO: 9 - DNA, 144 bp, Homo sapiens
cggtgataggtcgtattgtttcgcgagggttagaacgtttatttttgtagaggtgtatttaagtgatgtgtttgagtattgtttgagtgtatttttgtagaaatgggcgttttggttttcgcgaggtagtgcgatttgttatcg

SEQ ID NO: 10 - DNA, 63 bp, Artificial Sequence
  Primer for FMR1
  misc_feature (22)..(26): Wherein N = A, C, G or T
acatgcatgtcttcaaagtggnnnnnaggaggggcatgttctctcttcaagtggcctgggagc

SEQ ID NO: 11 - DNA, 21 bp, Artificial Sequence
  Primer binding site for FMR1
acatgcatgtcttcaaagtgg

SEQ ID NO: 12 - DNA, 24 bp, Artificial Sequence
  Primer for FMR1
tctctcttcaagtggcctgggagc

SEQ ID NO: 13 - DNA, 21 bp, Artificial Sequence
  Primer for FMR1
ccactttgaagacatgcatgt

SEQ ID NO: 14 - DNA, 23 bp, Artificial Sequence
  Primer for FMR1
ggatgcatttgatttcccacgcc

SEQ ID NO: 15 - DNA, 252 bp, Homo sapiens
ggatgcatttgagttcccacgccactgagtgcacctctgcagaaatgggcgttctggccctcgcgaggcagtgcgacctgtcaccgctcttcagccttcccgccctccaccaagcccgcgcacgcccggcccgcgcgtctgtctttcgacccggcacctcggccggttcccagcagcgcgcatgcgcgcgctcccaggccacttgaagagagaacatgcccctcctacaccccactttgaagacatgcatgt

SEQ ID NO: 16 - DNA, 67 bp, Artificial Sequence
  Primer for FMR1
  misc_feature (22)..(29): Wherein N = A, C, G or T
acatgcatgtcttcaaagtggnnnnnnncgattgtgcatgtcctctctcttcaagtggcctgggagc

SEQ ID NO: 17 - DNA, 67 bp, Artificial Sequence
  Primer for FMR1
  misc_feature (22)..(28): n is a, c, g, or t
acatgcatgtcttcaaagtggnnnnnnnctagtgtgcatgtcctctctcttcaagtggcctgggagc

SEQ ID NO: 18 - DNA, 65 bp, Artificial Sequence
  Primer for FMR1
  misc_feature (21)..(27): n is a, c, g, or t
tttgatagcggcctaaatcgnnnnnnngttatactatcaaatctctcttcaagtggcctgggagc

SEQ ID NO: 19 - DNA, 20 bp, Artificial Sequence
  Primer binding site for FMR1
tttgatagcggcctaaatcg

SEQ ID NO: 20 - DNA, 65 bp, Artificial Sequence
  Primer for FMR1
  misc_feature (35)..(41): n is a, c, g, or t
gtaccaacatgcatgtcttcaaagtggatggtacnnnnnnntctctcttcaagtggcctgggagc

SEQ ID NO: 21 - DNA, 26 bp, Artificial Sequence
  Primer for FMR1
cctctctcttcaaataacctaaaaac

SEQ ID NO: 22 - DNA, 25 bp, Artificial Sequence
  Primer for FMR1
gttgygggtgtaaatattgaaatta

(19) United States
(12) Patent Application Publication
(10) Pub. No.: US 2007/0020640 A1
(43) Pub. Date: Jan. 25, 2007

(54) Molecular encoding of nucleic acid templates for PCR and other forms of sequence analysis

(76) Inventors: Megan L. McCloskey, Seattle, WA (US); Charles D. Laird, Seattle, WA (US); R. Scott Hansen, Seattle, WA (US); Reinhard J. Stoger, Seattle, WA (US); Brooks Miner, Seattle, WA (US); Alice Burden, Seattle, WA (US)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(21) Appl. No.: 11/186,636

(22) Filed: Jul. 21, 2005

(51) Int. Cl. C12Q 1/68; G06F 19/00

(52) U.S. Cl. 435/6; 702/20

(57) ABSTRACT

In a first aspect, the present invention provides methods for authenticating a nucleic acid molecule and its sequence with a molecular barcode and batch-stamp. In another aspect, the present invention provides methods for authenticating a nucleic acid amplification product. In a further aspect, the present invention provides compositions for encoding both single-stranded and double-stranded target nucleic acids with coded oligonucleotides. The compositions are useful in the practice of the methods of the invention.

### STATEMENT OF GOVERNMENT LICENSE RIGHTS

The U.S. Government has a paid-up license in this invention and the right in limited circumstances to require the patent owner to license others on reasonable terms as provided for by the terms of ROI GM053805, P30 HD02274-3751, P30 HD002274-35S1, and HD 16659 awarded by National Institutes of Health.

### FIELD OF THE INVENTION

The present invention relates to the field of polymerase chain reaction (PCR) amplification of nucleic acid templates, and other forms of sequence analysis, particularly to the use of barcodes and batch-stamps for verifying the authenticity of PCR products and other sequence information.

### BACKGROUND OF THE INVENTION

The polymerase chain reaction (PCR) allows multiple copies of selected DNA sequences to be produced from limited amounts of a DNA template (Saiki et al., Science 230:1350-1354, 1985). Reactions with limited amounts of template, however, increase the risk of amplifying contaminant DNA and can also result in a skewed yield of PCR products such that there is a high degree of redundancy for a small portion of the original genomic sequences (Taylor et al., Pathology 29:309-312, 1997). Problems with contamination and redundancy are particularly pronounced in PCR reactions with rare and irreplaceable DNA templates.

There is a need in the art for methods for verifying the authenticity of PCR products. The present invention addresses this and other needs.

### SUMMARY OF THE INVENTION

In the practice of the present invention nucleic acid molecules (e.g., genomic DNA fragments) are labeled with distinct sequence tags prior to PCR amplification. These sequence tags authenticate a nucleic acid sequence with information, such as the date of the experiment, and the sample identity. Thus, the present invention permits identification of valid sequences, and distinguishes the valid sequences from contaminants and redundant sequences arising from template re-cloning. Contaminant sequences can be identified even when multiple control (no DNA) PCR samples are negative. Barcoding permits, for example, quantification of the relative abundance of genomic methylation patterns or polymorphic sequences by correcting for skewing that can arise from PCR amplification or the cloning of the products.

Examples of specific uses for the present invention include analysis of limited amounts of template DNA for biomedical, ancient DNA, and forensic purposes. For example, authentication of a forensic nucleic acid sample is crucial for comparison of samples from known and unknown individuals. The present invention provides a high level of confidence that a sample sequence represents a known sample processed on a certain day; contaminating sequences can be identified by their molecular sequence tags, or lack thereof, even when control (no DNA) PCR samples are negative.

A second example of a specific use for the present invention concerns methylation patterns. These methylation patterns are useful in diagnosis and prognosis of some cancers. The present invention provides methods to assess the variations of methylation, and their quantification, in a heterogeneous population of cancer cells and normal cells. A third example concerns ancient DNA, where samples generally have limited amounts of template. The present invention provides positive identification of sample DNA and of contaminants from analyzed sequences. A further example concerns mosaics in human disease. Some diseases (e.g., chronic myelogenous leukemia and scleroderma) are characterized by a small fraction of mosaicism of cancer cells, or of cells against which the host is making antibodies. The authentication of the mosaicism, and the quantification of the degree of mosaicism, are now possible using the present invention.

Accordingly, a first aspect of the invention provides methods for bar-coding a nucleic acid molecule. In some embodiments, the methods comprise the steps of (a) contacting a target nucleic acid molecule in a sample with a bar-coded oligonucleotide under suitable conditions to anneal the bar-coded oligonucleotide to the target nucleic acid molecule, wherein the bar-coded oligonucleotide comprises a first sequence complementary to the target nucleic acid molecule, a second sequence providing a random barcode, and a third sequence that is not complementary to any sequence in the sample; and (b) extending the annealed bar-coded oligonucleotide using the target nucleic acid molecule as a template to produce a bar-coded target nucleic acid molecule.

In another aspect, the present invention provides methods for authenticating a DNA amplification product comprising the steps of (a) contacting a target nucleic acid molecule in a sample with a bar-coded oligonucleotide under suitable conditions to anneal the bar-coded oligonucleotide to the target nucleic acid molecule, wherein the bar-coded oligonucleotide comprises a first sequence complementary to the target nucleic acid molecule, a second sequence providing a random barcode, and a third sequence that is not complementary to any sequence in the sample, and wherein the 5′ end of the bar-coded oligonucleotide comprises a tethering sequence that is complementary to a sequence 5′ to the first sequence; (b) extending the annealed bar-coded oligonucleotide using the target nucleic acid molecule as a template to produce a bar-coded target nucleic acid molecule; (c) amplifying the bar-coded target nucleic acid molecule using a primer that binds to the third sequence to produce an amplification product; and (d) authenticating the amplification product by detecting the presence of the second sequence in the amplification product.

In a further aspect, the present invention provides a method for authenticating a DNA amplification product comprising the steps of (a) ligating a hairpin linker to a double-stranded target DNA molecule to produce a ligated target DNA molecule, wherein the hairpin linker comprises a first sequence providing experimental identification information, a second sequence providing a random barcode comprising nucleotides selected from the group consisting of adenosines, guanidines, and thymidines, and a third sequence complementary to the first sequence; (b) treating the ligated target DNA molecule of step (a) under suitable conditions to convert cytosines in the ligated target DNA molecule to uracils; (c) amplifying the treated ligated target DNA molecule of step (b) to produce an amplification product; and (d) authenticating the amplification product of step (c) by detecting the presence of the first and second sequences in the amplification product.

In an additional aspect, the present invention provides compositions that each include a target nucleic acid molecule and a bar-coded oligonucleotide, wherein the bar-coded oligonucleotide comprises a first sequence complementary to the target nucleic acid molecule, a second sequence providing a random barcode, and a third sequence that is not complementary to any sequence in the sample, and wherein the 5′ end of the bar-coded oligonucleotide comprises a tethering sequence that is complementary to a sequence 5′ to the first sequence. The compositions are useful, for example, in the practice of the methods of the present invention.

### BRIEF DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 shows an exemplary bar-coded oligonucleotide of the invention containing a sequence complementary to the target nuclei acid molecules (1), a batch-stamp sequence (2), random barcode (3), a leftward primer binding site (4), and a 5′ tethering sequence (5), as described in EXAMPLES 1-5. The letter N represents a nucleotide randomly selected from A, T, C, or G.

FIGS. 2A-2D shows the steps in a representative method of the invention for authenticating a DNA amplification product. A: A target DNA molecule is denatured; B: the bar-coded oligonucleotide shown in FIG. 1 is annealed to one strand of the target DNA molecule; C: the bar-coded oligonucleotide is extended using Sequenase to synthesize a bar-coded target DNA molecule; D: the bar-coded target DNA molecule is amplified using the polymerase chain reaction (PCR). The closed circles represent polymerase molecules.

FIG. 3 shows a schematic representation of a bar-coded and batch-stamped hairpin linker (SEQ ID NO: 1), designed for ligation to DraIII-cut genomic DNA of FMR1 (SEQ ID NO:2), as described in EXAMPLE 6. The letter D represents a nucleotide randomly selected from A, G, and T.

FIGS. 4A-4G show FMR1 promoter sequences, with inferred methylation states of CpG sites, recovered from male fragile X patients using hairpin-bisulfite PCR with linker bar-coding and batch-stamping, as described in EXAMPLE 6. Unconverted (methylated) CpG dyads are black, and converted (unmethylated) CpG dyads are boxed. Within the 26 nt linker (boxed region at left), the randomized 7 nt variable barcodes are shaded at far left; the designated variable batch-stamps, which comprise 8 base pairs which end in A:T or T:A, are shaded at right. All sequences show 100% conversion of non-CpG cytosines. A: A distinctive hypermethylated sequence (SEQ ID NO:3); B, C: Redundant hypermethylated sequences recovered from independent bacterial colonies (SEQ ID NO:4 and SEQ ID NO:5), with identical barcodes and methylation patterns; D: A hypomethylated sequence with a distinctive barcode (SEQ ID NO:6); E, F: Redundant hypomethylated sequences with identical barcodes recovered from independent bacterial colonies (SEQ ID NO:7 and SEQ ID NO:8). These are distinguishable as redundant and as different from the hypomethylated sequence D only because of barcoding; G: A contaminant sequence bearing a hairpin linker that predates the addition of the barcode and batch-stamp, which was recovered during analysis of the same sample that generated sequences A to C (SEQ ID NO:9). Sequences A to C carry a different batch-stamp than sequences D to F, with the inversion of the A-T base pair, confirming that these sequence sets came from different DNA samples. Redundant hypermethylated sequences are denoted with asterisks (*), and redundant hypomethylated sequences with plus signs (+).

### DETAILED DESCRIPTION OF THE PREFERRED EMBODIMENT

A first aspect of the invention provides methods for bar-coding a nucleic acid molecule. In some embodiments, the methods comprise the steps of (a) contacting a target nucleic acid molecule in a sample with a bar-coded oligonucleotide under suitable conditions to anneal the bar-coded oligonucleotide to the target nucleic acid molecule, wherein the bar-coded oligonucleotide comprises a first sequence complementary to the target nucleic acid molecule, a second sequence providing a random barcode, and a third sequence that is not complementary to any sequence in the sample; and (b) extending the annealed bar-coded oligonucleotide using the target nucleic acid molecule as a template to produce a bar-coded target nucleic acid molecule. In some embodiments of the methods of this aspect of the invention, the 5′ end of the bar-coded oligonucleotide comprises a tethering sequence that is complementary to a sequence 5′ to the first sequence.

The term “target nucleic acid molecule” refers to a nucleic acid molecule that corresponds to a nucleic acid molecule that is to be bar-coded using the bar-coded oligonucleotides and methods of the invention. As used herein, a nucleic acid molecule “corresponds” to another nucleic acid molecule if it comprises a sequence that is identical to or complementary to the sequence of all or part of the other nucleic acid molecule. The term “nucleic acid molecule” encompasses both deoxyribonucleotides and ribonucleotides and refers to a polymeric form of nucleotides including two or more nucleotide monomers. The nucleotides can be naturally-occurring, artificial and/or modified nucleotides, or any other complementary subunits. Thus, the target nucleic acid molecule may be any nucleic acid molecule, including, but not limited to DNA and RNA (such as ribosomal RNA, messenger RNA, and small untranslated RNA). In some embodiments, the target nucleic acid molecule is a DNA molecule.

The term “sample” refers any specimen containing the target nucleic acid molecule that can be used for nucleic acid analysis, including, but not limited to, clinical samples, forensic samples, and ancient nucleic acid samples, and nucleic acid extracts prepared therefrom.

The bar-coded oligonucleotide comprises a first sequence that is complementary to the target nucleic acid molecule. The first sequence is located at the 3′ end of the bar-coded oligonucleotide. The length of the first sequence is sufficient to allow the oligonucleotide to anneal to the target nucleic acid molecule in order to prime the synthesis of a nucleic acid molecule that is complementary to the target nucleic acid molecule. In some embodiments, the length of the first sequence is between 18 and 30 nucleotides, such as between 20 and 29 nucleotides or between 22 and 25 nucleotides.

The bar-coded oligonucleotide further comprises a second sequence that provides a random barcode. The term “random barcode” refers to an arbitrary sequence that can uniquely identify a target nucleic acid in an experiment, and whose sequence is unknown at the start of the experiment. Methods of synthesizing oligonucleotides containing random sequences are well known in the art. Typically, oligonucleotides containing random sequences are synthesized by randomly incorporating a nucleotide at each of the positions of the second sequence. For example, a random nucleotide N may be selected from A, C, G, and T. In some embodiments, a random nucleotide D may be selected from A, G, and T. The second sequence is located 5′ to the first sequence in the bar-coded oligonucleotide. The length of the second sequence is sufficient to provide, with high probability, a unique identity to each target nucleic acid molecule in the sample prior to amplification. For example, a second sequence of 7 random nucleotides N selected from A, G, C, and T will provide a maximum of 4⁷ or 16,384 unique barcodes. In some embodiments, the length of the second sequence is between 3 and 30 nucleotides, such as between 5 and 25 nucleotides or between 7 and 13 nucleotides.

The bar-coded oligonucleotide further comprises a third sequence that is not complementary to any sequence in the sample. The third sequence serves as a primer binding site for the leftward primer for amplifying the bar-coded nucleic acid molecule, as further described below. The third sequence is located 5′ to the second sequence in the bar-coded oligonucleotide. The length of the third sequence is sufficient to provide a specific binding site for the leftward primer. In some embodiments, the length of the third sequence is between 18 and 30 nucleotides, such as between 20 and 29 nucleotides or between 22 and 25 nucleotides.

The 5′ end of the bar-coded oligonucleotide may contain a tethering sequence that is complementary to a sequence 5′ to the first sequence. The term “5′ tethering sequence” refers to a sequence that binds to an internal tethering sequence of the bar-coded oligonucleotide under suitable conditions. The term “internal tethering sequence” refers to the sequence of the bar-coded oligonucleotide that is complementary to the tethering sequence. The internal tethering sequence is located 5′ to the first sequence in the bar-coded oligonucleotide. For example, the internal tethering sequence may be located between the first sequence and the second sequence, or between the first sequence and the third sequence. The internal tethering sequence may also be between the first sequence and the fourth sequence (described below) or even a part of the fourth sequence.

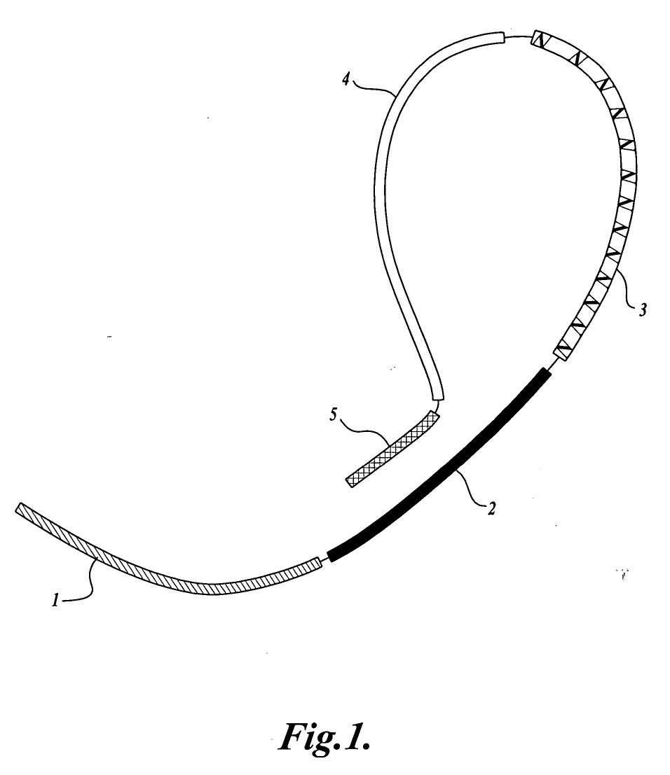

The binding of the 5′ tethering sequence to the internal tethering sequence produces a bar-coded oligonucleotide with a secondary structure in a stem-loop configuration, as shown in FIG. 1. In the stem-loop configuration of the bar-coded oligonucleotide, the tethering sequences form a double-stranded stem, the sequences between the first sequence and the 5′ end of the bar-coded oligonucleotide form a loop, and only the first sequence is outside the stem-loop structure. In the stem-loop configuration of the bar-coded oligonucleotide, the availability of sequences other than the first sequence to bind to nucleic acid sequences in the sample is greatly reduced.

The sequence composition (e.g., the G/C content) and length of the 5′ tethering sequence and the internal tethering sequence are selected to (1) enable the formation of a stable stem-loop structure of the bar-coded oligonucleotide, in which the tethering sequences are annealed and the leftward primer binding sites is unavailable for binding, under conditions in which the bar-coded oligonucleotide is annealed to the target nucleic acid molecule, and (2) to allow the stem-loop structure of the bar-coded oligonucleotide to melt sufficiently to make the leftward primer available for binding under conditions in which the bar-coded target nucleic acid molecule is amplified, as further described below. In some embodiments, the AG of the bar-coded oligonucleotide under conditions for annealing to the target nucleic acid molecule is less than −0.1, and the ΔG of the bar-coded oligonucleotide under conditions for amplifying the bar-coded target nucleic acid molecule is greater than 0.0. Methods for determining the ΔG of oligonucleotides are standard in the art. For example, the ΔG of an oligonucleotide may be determined using an algorithm that calculates the free energy of formation for various structures formed by sequences because of intra-strand base pairing, such as M-Fold (Zuker, Curr. Opin. Struct. Biol. 10:303-310, 2000) or DNA-fold (available at http://biocore.unl.edu/coreweb/dna-fold.htm). In some embodiments, the sequence composition and length of the 5′ tethering sequence and the internal tethering sequence are selected to provide a stable stem-loop structure that is stable at 37° C. and unstable at 63° C. In some embodiments, the length of the 5′ tethering sequence and the internal tethering sequence is between 2 and 10 nucleotides.

The 5′ tethering sequence may be a part of the binding site for the leftward primer, as described in EXAMPLES 1-3. However, in some embodiments, the 5′ tethering sequence is distinct from the third sequence to provide an internal monitor for re-bar-coding, as described in EXAMPLE 4. Re-bar-coding may occur when not all of the bar-coded oligonucleotide is removed between the step of bar-coding a target nucleic acid molecule and a subsequent step of amplifying the bar-coded target nucleic acid molecule. As a result, the bar-coded oligonucleotide primes the extension of a previously bar-coded nucleic acid molecule to produce a re-bar-coded nucleic acid molecule. Provided the tethering sequence is distinct from the third sequence, the 5′ tethering sequence will only be present in an amplified nucleic acid molecule if re-bar-coding occurred in the last round of amplification, and the absence of the 5′ tethering sequence in an amplification product indicates that no re-bar-coding during the last cycle had taken place.

In some embodiments, the bar-coded oligonucleotide further comprise a fourth sequence. The fourth sequence is a predetermined nucleic acid sequence providing a batch-stamp or experiment verification information. The phrase “predetermined nucleic acid sequence” means that the nucleic acid sequence of a nucleic acid molecule is previously known. The term “experimental identification information” or “batch-stamp” refers to information that uniquely identifies a specific experiment. For example, a batch-stamp may identify a sample, a patient, and/or an analysis date. The fourth sequence maybe located anywhere between the first sequence and the third sequence in the bar-coded oligonucleotide. The length of the fourth sequence is sufficient to provide a unique identity to the experiment. In some embodiments, the length of the fourth sequence is between 3 and 30 nucleotides, such as between 4 and 15 nucleotides or between 5 and 10 nucleotides.

In some embodiments an oligonucleotide may be used that does not depend on tethering, and therefore does not include a tethering sequence. These oligonucleotides would include a first, second, third and fourth sequence as described herein.

In step (a) of the methods of this aspect of the invention, a target nucleic acid molecule in a sample is contacted with a bar-coded oligonucleotide under conditions suitable to anneal the bar-coded oligonucleotide to the target nucleic acid molecule. Methods for annealing oligonucleotides are standard in the art. In the methods of the invention, the bar-coded oligonucleotide is annealed to the target nucleic acid molecule under conditions in which the bar-coded oligonucleotide is in a stable secondary stem-loop configuration (as a result of the binding of the 5′ tethering sequence to the internal tethering sequence), but the first sequence is linear and free to anneal to the target nucleic acid molecule. Suitable conditions for annealing a bar-coded oligonucleotide to a target nucleic acid molecule further include the presence at appropriate temperatures and for sufficient lengths of time of effective amounts of reagents, such as buffers, dithiothreitol, RNase inhibitors, and a deoxynucleotide triphosphate mixture. Typically, for oligonucleotides less than 100 bases in length, annealing conditions are 5° to 10° C. below the homoduplex melting temperature (Tₘ); see generally, Sambrook et al., Molecular Cloning: A Laboratory Manual, 2d ed., Cold Spring Harbor Press, 1989; Ausubel et al. Current Protocols in Molecular Biology, Greene Publishing, 1987). In some embodiments, the target nucleic acid molecule is contacted with the bar-coded oligonucleotide at a temperature between 1° C. and 70° C., such as at a temperature of about 37° C. Exemplary conditions suitable for annealing a bar-coded oligonucleotide to a target nucleic acid molecule are described in EXAMPLES 1-4.

In step (b) of the methods of this aspect of the invention, the annealed bar-coded oligonucleotide is extended using the target nucleic acid molecule as a template to produce a bar-coded target nucleic acid molecule. Methods for extending an annealed oligonucleotide using a nucleic acid molecule as a template are standard in the art. Suitable conditions for extending the annealed oligonucleotide producing a bar-coded target nucleic acid molecule include the presence at appropriate temperatures and for sufficient lengths of time of effective amounts of an enzyme and effective amounts of other reagents, such as buffers, dithiothreitol, RNase inhibitors, and a deoxynucleotide triphosphate mixture. Typically, the enzyme is a polymerase without exonuclease activity, such as Sequenase (USB). In some embodiments, the annealed bar-coded oligonucleotide is extended at a temperature between 1° C. and 70° C., such as at a temperature of about 37° C. Exemplary conditions suitable for extending an annealed bar-coded oligonucleotide to produce a bar-coded target nucleic acid molecule are described in EXAMPLES 1-4.

The bar-coded nucleic acid molecules produced by the methods of the first aspect of the invention may be amplified, as described below. In some embodiments, the bar-coded nuclei acid molecules may be directly sequenced without prior amplification (for example, using the method disclosed by Shendure J, et al., Nature Reviews of Genetics 5(5):335-344, May 2004).

A second aspect of the invention provides methods for authenticating a DNA amplification product. In some embodiments, the methods comprise the steps of (a) contacting a target nucleic acid molecule in a sample with a bar-coded oligonucleotide under suitable conditions to anneal the bar-coded oligonucleotide to the target nucleic acid molecule, wherein the bar-coded oligonucleotide comprises a first sequence complementary to the target nucleic acid molecule, a second sequence providing a random barcode, and a third sequence that is not complementary to any sequence in the sample, and wherein the 5′ end of the bar-coded oligonucleotide comprises a tethering sequence that is complementary to a sequence 5′ to the first sequence; (b) extending the annealed bar-coded oligonucleotide using the target nucleic acid molecule as a template to produce a bar-coded target nucleic acid molecule; (c) amplifying the bar-coded target nucleic acid molecule using a primer that binds to the third sequence to produce an amplification product; and (d) authenticating the amplification product by detecting the presence of the second sequence in the amplification product. Steps (a) and (b) of the methods of this aspect of the invention are as described for steps (a) and (b) of the methods of the first aspect of the invention.

In step (c) of the methods, the bar-coded target nucleic acid molecule is amplified using a primer that binds to the third sequence to produce an amplification product. In addition to the primer that binds to the third sequence (leftward primer), a primer that is complementary to the Sequenase product of the target nucleic acid molecule that is 3′, or upstream, to the first sequence is used (rightward primer), as shown in FIG. 1. Methods and conditions for amplifying nucleic acid molecules using the polymerase chain reaction (PCR) are standard in the art. Exemplary conditions for amplifying bar-coded target nucleic acid molecules are described in EXAMPLES 1-4 and 6.

In step (d) of the methods, the amplification product is authenticated by detecting the presence of the second sequence in the amplification product. Generally, the amplification products may be analyzed by gel electrophoresis, and further cloning and sequencing appropriately sized products. Methods and conditions for analyzing nucleic acid molecules by gel electrophoresis, cloning, and sequencing are standard in the art. Exemplary conditions for analyzing bar-coded target nucleic acid molecules by gel electrophoresis, cloning, and sequencing are described in EXAMPLES 1-4 and 6.

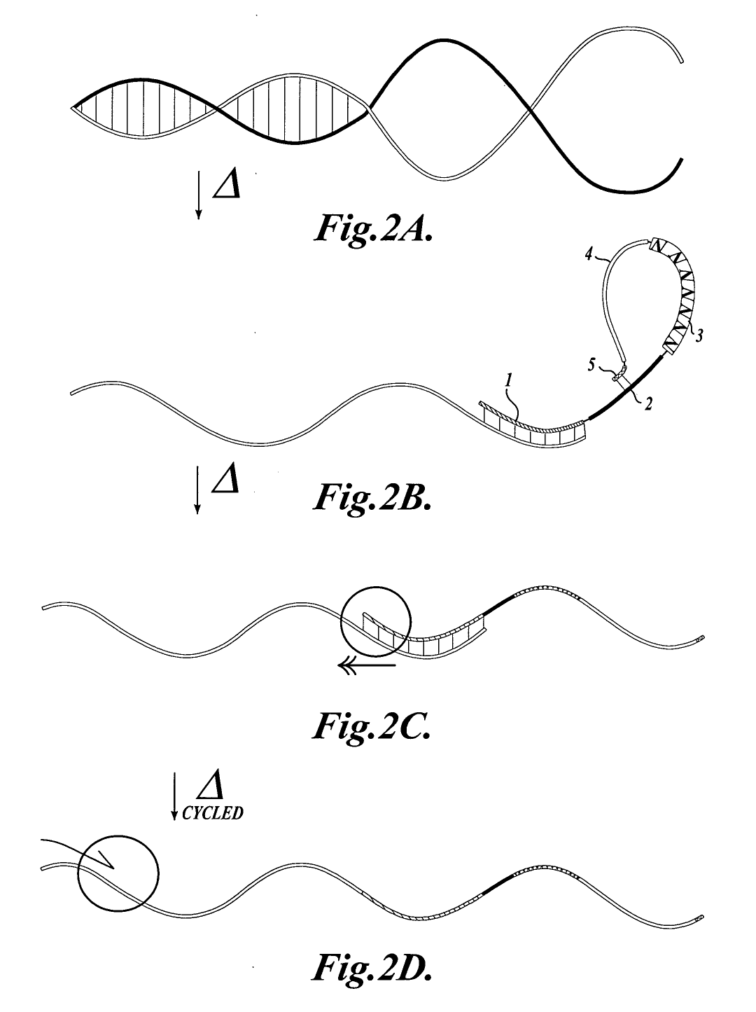

In some embodiments, the bar-coded oligonucleotide further comprise a fourth sequence, as described above for the first aspect of the invention, and step (d) further comprises authenticating the amplification product by detecting the presence of the fourth sequence in the amplification product. Thus, some embodiments of the methods of the invention comprise the steps of (a) contacting a target nucleic acid molecule in a sample with a bar-coded oligonucleotide under suitable conditions to anneal the bar-coded oligonucleotide to the target nucleic acid molecule, wherein the bar-coded oligonucleotide comprises a first sequence complementary to the target nucleic acid molecule, a second sequence providing a random barcode, a third sequence that is not complementary to any sequence in the sample, and a fourth sequence providing experimental identification information, and wherein the 5′ end of the bar-coded oligonucleotide comprises a tethering sequence that is complementary to a sequence 5′ to the first sequence; (b) extending the annealed bar-coded oligonucleotide using the target nucleic acid molecule as a template to produce a bar-coded target nucleic acid molecule; (c) amplifying the bar-coded target nucleic acid molecule using a primer that binds to the third sequence to produce an amplification product; and (d) authenticating the amplification product by detecting the presence of the second sequence and the fourth sequence in the amplification product. A representative method of this aspect of the invention is schematically illustrated in FIG. 2.

A third aspect of the invention provides methods for authenticating a DNA amplification product. In some embodiments, the methods comprise the steps of (a) ligating a hairpin linker to a double-stranded target DNA molecule, wherein the hairpin linker comprises a first sequence, a second sequence, and a third sequence, wherein the hairpin linker comprises a first sequence providing experimental identification information, a second sequence providing a random barcode comprising nucleotides selected from the group consisting of adenosines, guanidines, and thymidines, and a third sequence complementary to the first sequence; (b) treating the ligated target DNA molecule of step (a) under suitable conditions to convert cytosines in the ligated target DNA molecule to uracils; (c) amplifying the treated ligated target DNA molecule of step (b) to produce an amplification product; and (d) authenticating the amplification product of step (c) by detecting the presence of the second sequence in the amplification product. In embodiments of this aspect of the invention wherein a hairpin linker is ligated to a double-stranded target DNA molecule, the stem of the hairpin is tethered and self complementary to ensure that there is a double stranded form that can be ligated with digested double strand DNA.

In step (a) of these methods, a hairpin linker is ligated to a digested, double-stranded, target DNA molecule. The hairpin linker comprises a first sequence providing experimental identification information. The first sequence providing experimental identification information is as described above, for the bar-coded oligonucleotides used in the first and second aspects of the methods of the invention.

The hairpin linker further comprises a second sequence providing a random barcode. The second sequence providing the random barcode is as described above for the bar-coded oligonucleotides used in the first and second aspects of the methods of the invention, except that it only comprises nucleotides selected from the group consisting of adenosines, guanidines, and thymidines, and a third sequence complementary to the first sequence.

The third sequence in the hairpin linker is complementary the second sequence, to provide a hairpin secondary structure for the linker, in which the first and third sequences are annealed, and the second sequence forms a loop. This represents an additional form of tethering to ensure proper secondary structure required for ligation to genomic DNA.

In this aspect of the invention, the digestion of the double-strand DNA provides a defined overhang that allows for specific ligation. The overhang is determined by the enzyme used and the locus that is targeted for amplification. Blunt-ended hairpin ligation may also be implemented.

A fourth aspect of the invention provides composition comprising a target nucleic acid molecule and a bar-coded oligonucleotide, wherein the bar-coded oligonucleotide comprises a first sequence complementary to the target nucleic acid molecule, a second sequence providing a random barcode, and a third sequence that is not complementary to any sequence in the sample, and wherein the 5′ end of the bar-coded oligonucleotide comprises a tethering sequence that is complementary to a sequence 5′ to the first sequence. The first, second, and third sequences, and the tethering sequences are as described above. In some embodiments, the bar-coded oligonucleotide further comprises a fourth sequence providing experimental identification information, as also described above.

The following examples illustrate representative embodiments now contemplated for practicing the invention, but should not be construed to limit the invention.

### EXAMPLE 1

This example describes an exemplary method of the invention for authenticating a PCR product from the FMR1 locus by using a bar-coded oligonucleotide with a 5-nucleotide random barcode.

1. Materials and Methods

Target DNA: Human genomic DNA was isolated from blood using a standard protocol. DNA samples were obtained from blood treated with proteinase K, then recovered using a phenol extraction. Isolated DNA was resuspended in TE buffer and stored at −20° C.

Bar-coded Oligonucleotide: Oligo HBP (5′ ACATGCATGTCTTCAAAGTGG NNNNN AGGAGGG GCATGT TCTCTCTTCAAGTGGCCTGGGAGC 3′, SEQ ID NO:10). From 5′ to 3′, oligo HBP contains a unique, non-genomic sequence (5′ ACATGCATGTCTTCAAAGTGG 3′, SEQ ID NO:11) that provides a leftward primer binding site (region (4) in FIG. 1); a 5-nucleotide random barcode (NNNNN; region (3) in FIG. 1); a batch-stamp (5′ AGGAGGG 3′; region (2) in FIG. 1); an internal tethering sequence (5′ GCATGT 3′) that is complementary to the first 6 nucleotides of the leftward primer binding site (i.e., complementary to region (5) in FIG. 1); and a 24-nucleotide sequence complementary to FMR1 (5′ TCTCTCTTCAAGTGGCCTGGGAGC, SEQ ID NO: 12; region (1) in FIG. 1), a single copy gene on the X chromosome.

Oligo Annealing and Extension: Oligo HBP was diluted to 450 nM in 1× Sequenase buffer (US Biochemical), and heated at 95° C. for 5 minutes. The diluted oligo was then allowed too cool gradually to 4° C. In a separate tube 0.25 micrograms of genomic DNA was added to 1.65 microliters of 5× Sequenase buffer, and the total DNA mixture was brought up to a final volume of 13 microliters. This mixture was heated at 95° C. for 2 minutes to denature the DNA. After denaturation, 2 microliters of the pre-diluted oligo HBP was added, bringing the volume to 15 microliters. This mixture was then heated at 37° C. for 15 minutes. During this time a fresh mixture of Mg-dNTP was made. The Mg-dNTP mixture has the following concentrations: 20 mM MgCl₂, 2.0 mM DTT, 0.25 mM dNTPs, and 1× Sequenase buffer. After the 37° C. incubation was complete, 2.0 microliters of the freshly prepared Mg-dNTP mixture was added, followed by 1.5 microliters of 1:5 diluted Sequenase v2.0 (US Biochemical) in TE buffer. The final volume was 18.5 microliters. The mixture was then incubated at 37° C. for 15 minutes. This incubation was followed by a further incubation at 67° C. for 15 minutes to reduce the activity of the Sequenase. These steps allow for the extension of the annealed oligo onto the genomic template, resulting in a bar-coded template for subsequent amplification. The mixture was then stored at 4° C. until PCR.

PCR Reaction: 10 micoliters of the post-Sequenase products were used in a 100 microliters Hotstar Master Mix (Qiagen) that also had 2 microliters of each of the 50 micromolar leftward primer (5′CCACTTTGAAGACATGCATGT 3′, SEQ ID NO:13) and rightward primer (5′ GGATGCATTTGATTTCCCACGCC 3′, SEQ ID NO:14). PCR was initiated at 95° C. for 15 minutes per the manufacturer's instructions, and cycled in the following way: 95° C. for 30 seconds, annealed at 61° C. for 30 seconds, and extended at 72° C. for 40 seconds; 35 cycles were run.

Screening of Bar-coded PCR Products: The resulting band of the appropriate length of bar-coded FMR1 products was recovered from a 1.8% agarose gel visualized with ethidium bromide. The DNA was purified from the slice with the Qiagen PCR Purification kit for agarose gels, per manufacturer's instructions. Isolated encoded DNA was then transformed into chemically competent E. coli (Invitrogen) per manufacturer's instructions. Isolated colonies were then picked for a screening PCR(SPCR). A portion of the SPCR reactions were visualized on an agarose gel to verify the presence of the transformed vector. Verified SPCR reactions were then cleaned with Microclean (Gel Company) and sequenced on an ABI 3100 with Big Dye.

2. Results and Discussion

A representative amplified product, MM22, highlights the confirmed bar-coded regions:

| | |
|---|---|
| MM22: | |
| 5′ggatgcatttgagttcccacgccactgagtgca | (SEQ ID NO:15) |
| cctctgcagaaatgggcgttctggccctcgcgaggc | |
| agtgcgacctgtcaccgctcttcagccttcccgccc | |
| tccaccaagcccgcgcacgcccggcccgcgcgtctg | |
| tctttcgacccggcacctcggccggttcccagcagc | |
| gcgcatgcgcgc GCTCCCAGGCCACTTGAAGAGAG | |
| A ACATGCCCCTCCT ACACCC CACTTTGAAGACA | |
| TGCATGT 3′ | |

The sequences in lower case is the region complementary to FMR1 that was not represented in the bar-coded oligonucleotide, starting with the rightward primer binding site (italicized). The sequences in upper case corresponds to the sequence of the bar-coded oligonucleotide, starting with the reverse complement of the sequence complementary to FMR1 used to anneal the bar-coded oligonucleotide (i.e., the reverse complement of 5′ TCTCTCTTCAAGTGGCCTGGGAGC, SEQ ID NO:12), followed by the six nucleotides corresponding to the internal tether (5′ ACATGC 3′), the seven nucleotides corresponding to the batch-stamp (5′CCTCCT 3′), the 5 nucleotides corresponding to the barcode (5′ ACACCC 3′), and the leftward primer binding site (italicized).

132 sequences were obtained from this experiment. There were 22 sequences with a barcode that was identical to a sequence already obtained (i.e., redundant sequences). The remaining 110 sequences had distinct barcode regions that were 5 nucleotides long, indicating that those sequences originated from separate cells, or separate genomic target molecules. As expected, because this was the first assay of its kind, there was no detectable contamination. All recovered sequences had the proper batch-stamp for oligo HBP (5′CCCTCCT 3′). The ability to distinguish redundant from valid data is an immensely powerful tool.

### EXAMPLE 2

This example describes an exemplary method of the invention for authenticating a PCR product from the FMR1 locus by using a bar-coded oligonucleotide with a 7-nucleotide random barcode.

1. Materials and Methods

Target DNA: Human genomic DNA was isolated as described in EXAMPLE 1.

Bar-coded Oligonucleotide: Oligo MLM1 (5′ ACATGCATGTCTTCAAAGTGG NNNNNNN CGATTGT GCATGT CCTCTCTCTTCAAGTGGCCTGGGAGC 3′, SEQ ID NO: 16). From 5′ to 3′, oligo MLM1 contains a unique, non-genomic sequence (5′ACATGCATGTCTTCAAAGTGG 3′, SEQ ID NO:11) that provides a leftward primer binding site (region (4) in FIG. 1); a 7-nucleotide random barcode (NNNNNNN; region (3) in FIG. 1); a batch-stamp (5′CGATTGT 3′; region (2) in FIG. 1); an internal tethering sequence (5′ GCATGT 3′) that is complementary to the first 6 nucleotides of the leftward primer binding site (i.e., complementary to region (5) in FIG. 1); and a 24-nucleotide sequence complementary to FMR1 (region (1) in FIG. 1). A bar-code sequences of 7 random nucleotides provides the possibility of 47 distinct sequences.

Oligo Annealing and Extension: Oligo MLM1 was denatured, annealed to genomic DNA, and extended as described in EXAMPLE 1.

Purification of Extension Products: Unless the unincorporated oligo is removed after extension, it is possible that unincorporated oligo could be used as a primer in subsequent PCR reactions. This could lead to re-bar-coding of products and contaminants. To avoid this, post-Sequenase products were cleaned using QIAQuick PCR purification columns (Qiagen) according to manufacturer's instructions. Because the MLM1 oligo is longer than the tested lengths that Qiagen provides information for, the exact efficiency or removal of unincorporated oligo was not determined at this time.

PCR Reaction: 10 micoliters of the purified post-Sequenase products were used in a 100 microliters Hotstar Master Mix (Qiagen) that also had 2 microliters of each of the 50 micromolar leftward primer (5′CCACTTTGAAGACATGCATGT 3′, SEQ ID NO:13) and rightward primer (5′ GGATGCATTTGATTTCCCACGCC 3′, SEQ ID NO:14). PCR was initiated at 95° C. for 15 minutes per the manufacturer's instructions, and cycled in the following way: 95° C. for 30 seconds, annealed on a temperature block ranging from 60.8° C. to 64.1° C. for 30 seconds, and extended at 72° C. for 40 seconds; 35 cycles were run.

Screening of Bar-coded PCR Products: Bar-coded FMR1 products were recovered and screened as described in EXAMPLE 1.

2. Results and Discussion

46 sequences were obtained from this experiment. The barcodes, batch-stamps, and the annealing temperature of four exemplary bar-coded sequences are shown in TABLE 1. 36 of the 46 were desirable and valid sequences, each of which had a unique barcode. Only one of the 46 sequences was a contaminant sequence: it contained the batch-stamp of the HBP oligo used in EXAMPLE 1 and a 5-nculeotide barcode (MLM1_{—}43 in Table 1). A redundant sequence was also recovered, meaning that a MLM1-positive sequence with an already seen bar-code was recovered. One of these two sequences was counted as a valid sequence. The remaining 8 of the 46 sequences were the result of the kind of PCR error and non-specific amplification that is usually observed prior to temperature optimization of a PCR reaction. They also suggest that re-bar-coding is a low-probability event due to the fact that a contaminant was able to make it through without being re-bar-coded by an excess amount of MLM1 oligo.

TABLE 1
Exemplary Bar-Coded PCR Products

| | Annealing | | |
|---|---|---|---|
| Sequence Name | Temperature | Batch-Stamp | Bar Code |
| MLM1_22 | 63.5° C. | ACAATCG | TGGGCGA |
| MLM1_23 | 60.8° C. | ACAATCG | CAAATCA |
| MLM1_43 | 60.8° C. | CCCTCCT | ACCAG |
| MLM1_30 | 63.5° C. | ACAATCG | CAAATCA |

Sequences MLM1_{—}22 and MLM1_{—}30 have the barcode and they were produced using the same annealing temperature (63.5° C.), which was much higher than the annealing temperature that produced the contaminant MLM1_{—}43 (60.8° C.). This may suggest that the PCR reaction will have a higher specificity at higher temperatures for annealing the primers.

### EXAMPLE 3

This Example describes an exemplary method of the invention for authenticating a PCR product from the FMR1 locus by using two bar-coded oligonucleotides, each with a 7-nucleotide random barcode.

1. Materials and Methods

Target DNA: Human genomic DNA was isolated as described in EXAMPLE 1.

Bar-coded Oligonucleotides: Oligo MLM3 (5′ACATGCATGTCTTCAAAGTGG NNNNNNN CTAGTGT GCATGT CCTCTCTCTTCAAGTGGCCTGGGAGC 3′, SEQ ID NO:17). From 5′ to 3′, oligo MLM3 contains a unique, non-genomic sequence (5′ ACATGCATGTCTTCAAAGTGG 3′, SEQ ID NO:11) that provides a leftward primer binding site (region (4) in FIG. 1); a 7-nucleotide random barcode (NNNNNNN; region (3) in FIG. 1); a batch-stamp (5′CTAGTGT 3′; region (2) in FIG. 1); an internal tethering sequence (5′ GCATGT 3′) that is complementary to the first 6 nucleotides of the leftward primer binding site (i.e., complementary to region (5) in FIG. 1); and a 24-nucleotide sequence complementary to FMR1 (region (1) in FIG. 1), a single copy gene on the X chromosome.

Oligo CBP1 (5′ TTTGATAGCGGCCTAAATCG NNNNNNN GTTATACT ATCAAA TCTCTCTTCAAGTGGCCTGGGAGC 3′, SEQ ID NO:18). From 5′ to 3′, oligo MLM3 contains a unique, non-genomic sequence (5′ TTTGATAGCGGCCTAAATCG 3′, SEQ ID NO:19) that provides a leftward primer binding site (region (4) in FIG. 1); a 7-nucleotide random barcode (NNNN; region (3) in FIG. 1); a batch-stamp (5′ GTTATACT 3′; region (2) in FIG. 1); an

internal tethering sequence (5′ ATCAAA 3′) that is complementary to the first 6 nucleotides of the leftward primer binding site; region (5) in FIG. 1); and a 24-nucleotide sequence complementary to FMR1 (5′ TCTCTCTTCAAGTGGCCTGGGAGC, SEQ ID NO:12; region (1) in FIG. 1).

Oligo Annealing and Extension: Oligo MLM3 and CBP1 were denatured as described in EXAMPLE 1. Oligo MLM1 was annealed to genomic DNA, and extended as described in EXAMPLE 1. This was done in tandem 4 times. The post-Sequenase products were divided into three samples: (1) 24 microliters was purified and amplified as described in Example 2 below (“normal samples”); (2) 50 microliters was “spiked” with a concentration of oligo CBP1 that was equal to the concentration of MLM3 (i.e., 5.4 microliters of the 490 nM stock). 25 microliters of the spiked reaction mix was purified and amplified as described in Example 2 (“spiked sample”); and (3) 25 microliters of the spiked reaction mix was amplified as described below, without purification (“dirty spiked sample”).

Purification of Extension Products: Post-Sequenase products (normal sample and spiked sample) were cleaned as described in EXAMPLE 2.

PCR Reaction: 10 micoliters of the purified post-Sequenase products were used in a 100 microliters Hotstar Master mix (Qiagen) that included 2 microliters of each of the 50 micromolar leftward primer and the rightward primer. Two separate PCR reactions were run in parallel with each sample, one with the MLM3 leftward primer and one with the CBP1 leftward primer so as to avoid primer interference. PCR was initiated at 95° C. for 15 minutes per the manufacturer's instructions, and cycled in the following way: 95° C. for 30 seconds, annealed on a temperature block ranging from 61.4° C. to 64.1° C. for 30 seconds, and extended at 72° C. for 40 seconds; 35 cycles were run.

Screening of Bar-coded PCR Products: Bar-coded FMR1 products were recovered and screened as described in EXAMPLE 1.

2. Results and Discussion

107 sequences were obtained from this experiment. The results were interpreted as follows. The addition of a second primer binding site via a second oligo, tests the efficiency of colum removal, and it also tests for biased PCR amplification as the PCR reactions only have one leftward primer. Thus, PCR reactions with the CBP1 leftward primer will preferentially amplify the sequences containing the CBP1 oligo. As expected, equal amounts of oligos were present in the dirty samples which had not been column purified. It was also expected that in the normal samples there would be no CBP1 annealed sequences available. In addition, the reactions containing the CBP1 leftward primer were less efficient, whether or not the sample was purified. This was apparent as the bands produced from such amplifications were faint when visualized on an agarose gel. The reaction of the normal sample with MLM3 leftward primer produced a distinct and more visible band. No band was produced form the normal sample reactions using the CBP1 leftward primer. The spiked sample reaction using the MLM3 leftward primer produced a bright and distinct band (this also may indicate that the product was concentrated during the column purification). However, the spiked sample reaction using the CBP1 leftward primer produced a band that was very faint.

These experiments demonstrate that two different bar-coded oligos can be used in a reaction and differentially amplified using different primers.

### EXAMPLE 4

This Example describes an exemplary method of the invention for authenticating a PCR product from the FMR1 locus by using a bar-coded oligonucleotide with a 7-nucleotide random barcode and with a 5′ tethering sequence that is 5′ to the leftward primer binding site.

1. Materials and Methods

Target DNA: Human genomic DNA was isolated as described in EXAMPLE 1.

Bar-coded Oligonucleotide: Oligo MLM12 (5′ GTACCA ACATGCATGTCTTCAAAGTGG ATGGTAC NNNNNNN TCTCTCTTCAAGTGGCCTGGGAGC 3′, SEQ ID NO:20). From 5′ to 3′, oligo MLM1 contains a 6-nucleotide 5′ tethering sequence (5′ GTACCA 3′; region (5) in FIG. 1) that is complementary to the batch-stamp sequence; a unique, non-genomic sequence (5′ ACATGCATGTCTTCAAAGTGG 3′, SEQ ID NO:11) that provides a leftward primer binding site (region (4) in FIG. 1); a batch-stamp (5′ ATGGTAC 3′; region (2) in FIG. 1); a 7-nucleotide random barcode (NNNNNNN; region (3) in FIG. 1); and a 24-nucleotide sequence complementary to FMR1 (5′ TCTCTCTTCAAGTGGCCTGGGAGC, SEQ ID NO:12; region (1) in FIG. 1).

Oligo Annealing and Extension: Oligo MLM12 was denatured, annealed to genomic DNA, and extended as described in EXAMPLE 1.

Purification of Extension Products: A third of the post-Sequenase products were cleaned using QiaQuick PCR purification columns (Qiagen) according to manufacturer's instructions, a third were cleaned with the Strateprep PCR purification kit (Stratagene) according to manufacturer's instructions, and a third were not purified.

PCR Reaction: 2.5 micoliters of the post-Sequenase products were used in a 25 microliters Hotstar Master Mix (Qiagen) that also had 0.5 microliters of each of the 50 micromolar leftward primer (5′CCACTTTGAAGACATGCATGT 3′, SEQ ID NO:13) and rightward primer (5′ GGATGCATTTGATTTCCCACGCC 3′, SEQ ID NO:14). PCR was initiated at 95° C. for 15 minutes according to manufacturer's instructions, and cycled in the following way: 95° C. for 30 seconds, annealed at 63.5° C. for 30 seconds, and extended at 72° C. for 40 seconds; 21, 23, 25, 27, 29, 31, 33, and 35 cycles were run separately to collect data in a real-time manner. In tandem, a PCR reaction for each sample was performed without a leftward primer. This lack of leftward primer forces any excess bar-coded oligo to behave as a primer if it is able to do so. Therefore, this presents a direct assay for re-bar-coding. If the bar-coded oligo is used as a primer, then a band will be produced and the 6 bases 5′ of the primer binding (the 5′ tethering sequence, 5′ GTACCA 3′) will be present in the amplified sequences. If the oligo is used properly, as in the experiments with a leftward primer, then the extra bases will not be present in any amplified sequences as they are 5′ of the leftward primer binding site.

Screening of Bar-coded PCR Products: Bar-coded FMR1 products were recovered and screened as described in EXAMPLE 1.

2. Results and Discussion

Results showed there was absolutely no visible band produced from reactions with purified samples and without a leftward primer. The data indicate that the efficiency of the column purification is high. The extra 6 nucleotides 5′ of the leftward primer binding site act as an elegant and simple internal control for re-bar-coding during the final PCR cycle. The reactions without a leftward primer are more efficient than a water blank in this case for detecting contamination and re-bar-coding. Sequences obtained from this experiment will estimate and monitor the frequency of re-barcoding in the final cycle of PCR amplification.

### EXAMPLE 5

This Example describes an exemplary method of the invention for bar-coding a target RNA molecule by using a bar-coded oligonucleotide.

The design of barcode oligonucleotides for bar-coding a target RNA molecule will encompass the same concepts as described in EXAMPLES 1-4 one. The bar-coded oligonucleotide contains a region complementary to the target RNA strand. The bar-coded oligonucleotide also contains a batch-stamp region, a barcode region, and a unique primer binding site, as described in EXAMPLES 1-4.

The bar-coded oligonucleotide is used in a reverse transcriptase reaction as is standard in the art. After one round of reverse transcription, the reaction is stopped and the mixture is digested with an appropriate amount of uracil glycosylase to destroy any remaining RNA template (which includes uracil) in the mix. The digested mixture is then column-purified, as previously described. Post column-purification standard PCR is carried out with cycles 2-35 cycles. The screening of bar-coded PCR products is performed as described in EXAMPLE 1.

The ability to digest uracil-containing oligonucleotides with uracil glycosylase provides the opportunity to design oligos with uracil to further reduce the possibility of recoding with excess oligonucleotide. To utilize this opportunity, the oligonucleotides may contain uracil, replacing some of the thymines. In this embodiment of the invention, Sequenase extension (EXAMPLES 1-4) or reverse transcriptase extension (described in this Example), is carried out as described above. In this embodiment of the invention, the digestion with uracil glycosylase occurs after one round of PCR, thus fragmenting remaining uracil containing oligonucleotides. This technique may be preferable for applications requiring a higher degree of certainty that rebarcoding will not occur.

### EXAMPLE 6

This Example describes an exemplary method of the invention for bar-coding a PCR product from the FMR1 locus in a hairpin-bisulfite PCR reaction with a double-stranded DNA template (Miner et al., Nucl. Acids Res. 32(17):e135, 2004, herein incorporated by reference).

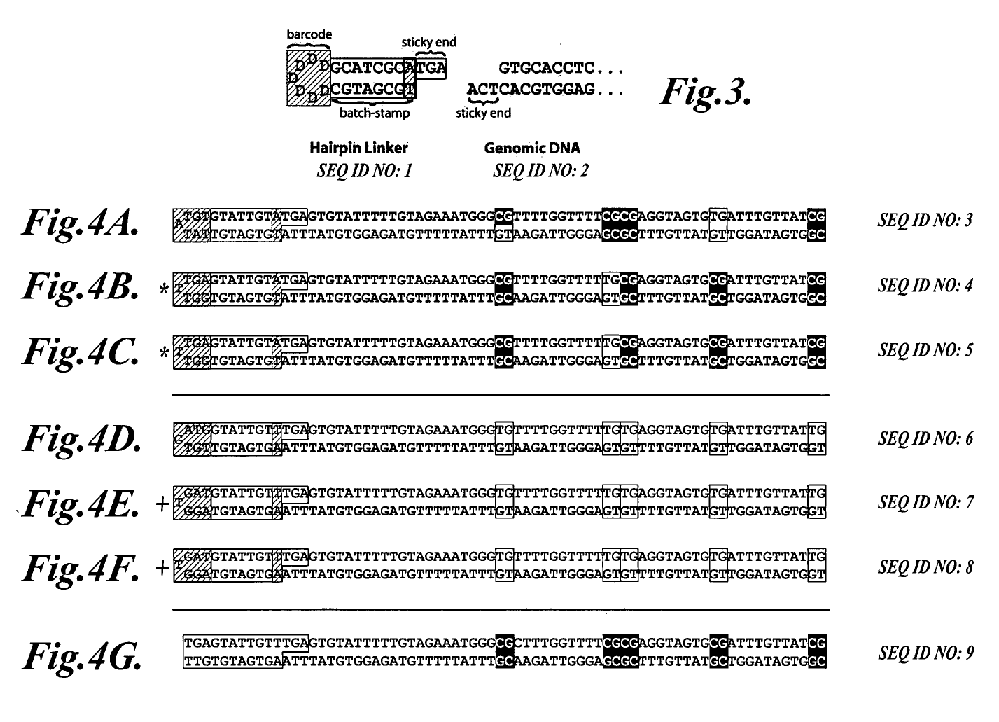

There is an increased risk of redundancy and contamination when amplifying limited amounts of template DNA, for example, when the goal is to compare and quantify sequences from different cells represented in the same DNA sample, as in bisulfite methylation analysis (Stoeger et al., Hum. Mol. Genet. 6:1791-1801, 1997). The frequent observation of multiple amplified sequences derived from a single original molecule was also noted in the context of bisulfite genomic sequencing, a method increasingly used in epigenetic research (Millar et al., Methods 27:108-113, 2002). In response to the challenges of PCR redundancy and contamination associated with PCR amplification of limited amounts of DNA template, genomic DNA fragments were labeled with molecular sequence barcodes and “batchstamps” prior to PCR amplification by including these molecular labels in the hairpin linker sequence (FIG. 3) that is used in hairpin-bisulfite PCR (Laird et al., Proc. Natl. Acad. Sci. USA 101:204-209, 2004), as described below. This encoded information enables the genomic origin of each sequence obtained from PCR and subsequent bacterial cloning to be tracked. Each genomic fragment is marked prior to amplification, allowing us to identify contaminant and redundant sequences and to quantify accurately the proportion of cells carrying a particular sequence variant by counting only distinctly tagged sequences. This highly sensitive method offers confirmation of the independent genomic origin of all sequences in final data sets derived from PCR amplification.

1. Materials and Methods

Conditions for hairpin-bisulfite PCR of human genomic FMR1 sequences (Laird et al., Proc. Natl. Acad. Sci. USA 101:204-209, 2004) were as follows: 5 μg of genomic DNA was cleaved by 10 U each of restriction endonucleases DraIII and AluI for 1 h at 37° C., followed by enzyme inactivation at 65° C. for 20 min. The use of a second restriction endonuclease, in this case AluI, removed the CG-rich sequence distal to the region analyzed. Ligation of the hairpin linker (5′ AGC-GATGCDDDDDDDGCATCGCT-TGA 3′, SEQ ID NO:1) with variations in the non-random nucleotides for batch-stamps) to DraIII-cleaved genomic DNA was for 15 min at 20° C., using 400 U of T4 ligase in 20 μl with 1× ligation buffer (New England Biolabs), followed by enzyme inactivation at 65° C. for 20 min.

The bisulfite conversion followed a previously published protocol (Laird et al., Proc. Natl. Acad. Sci. USA 101:204-209, 2004) with additional thermal denaturation steps. Hairpin-ligated DNA was denatured in 0.3M NaOH for 20 min, then heated to 100° C. for 1 min before addition of sodium bisulfite and hydroquinone to 3.4 M and 1 mM, respectively. The reaction mixture was incubated for 6 h at 55° C., with additional thermal denaturation steps (99° C. for 90 s, 10 times over the 6 h), and then incubated for an additional 6 h at 55° C. This was followed by a purification step using QIAquick PCR purification columns (Qiagen), subsequent treament with NaOH (final concentration 0.3 M) at 37° C. for 20 min, and another purification using Microspin S-200 HR columns (Amersham Pharmacia Biosciences).

PCR conditions were Hotstar Master Mix (Qiagen), with denaturation at 95° C. for 15 min, followed by 38 cycles of denaturing at 95° C. for 30 s, annealing at 58° C. for 30 s, and extension at 72° C. for 45 s; this was followed by a final extension at 72° C. for 5 min. Primers used were (i) first primer, 5′-CCTCTCTCTTCAAATAACCTAAAAAC-3′ (SEQ ID NO:21) and (ii) second primer, 5′-GTTGYGGGTGTAAATATTGAAATTA-3′ (SEQ ID NO:22).

All PCR products were analyzed by agarose gel electrophoresis; further cloning and sequencing of appropriately sized products was with TOPO TA Cloning Kits (Invitrogen Life Technologies); sequencing reactions were carried out with fluorescent dideoxy nucleotides (BIGDYE Terminator 3.1, Applied Biosystems), at either the DNA Sequencing Facility, Department of Biochemistry, or the Comparative Genomics Center, Department of Biology, University of Washington. Each sequence was proofread against the sequence trace; errant base calling was corrected manually before being presented here. For purposes of analysis and presentation, the output sequence was folded, using word processing software, into a hairpin conformation so that both strands aligned.

Results

The challenge of amplifying limited amounts of DNA template can result from trace amounts of initial DNA sample, or from laboratory analyses that include substantial DNA degradation as a necessary side effect of processing, as in bisulfite genomic sequencing (Grunau et al., Nucl. Acids Res. 29:e65, 2001). One of the major problems encountered in these analyses is to capture accurately the genomic template diversity following the steps of PCR and bacterial cloning. Hairpin-bisulfite PCR involves the ligation of a synthetic hairpin linker to the ends of a double-stranded genomic DNA fragment prior to bisulfite conversion and PCR amplification (Laird et al., Proc. Natl. Acad. Sci. USA 101:204-209, 2004). While the primary purpose of the hairpin linker is to maintain attachment of complementary strands, it can also be used to encode each ligated genomic fragment with information that distinguishes it from other sequences within a sample, allowing the evaluation of cloned sequences for redundancy and contamination. To accomplish this, the 6 nt loop of a hairpin linker (Laird et al., Proc. Natl. Acad. Sci. USA 101:204-209, 2004) was replaced with 7 nt randomly selected from A, G, and T. Cytosine was not used because its identity would be ambiguous after bisulfite conversion. With a random 7 nt barcode, the number of possible codes is 2187; in selecting 15 cloned PCR products from one DNA sample, the probability that two of these will be different genomic fragments labeled with identical 7 nt barcodes is 0.047 (for details of this probability calculation, see Miner et al., Nucl. Acids Res. 32(17):e135, 2004, Supplementary Materials).

Some applications will require a larger pool of random-sequence barcodes if more independently derived sequences are required. Linkers with up to 13 nt in the hairpin loop have been used with no observable detriments to sequence recovery. A 13 nt barcode gives ˜1.6×10⁶ different codes; even for a selection of 100 cloned PCR products, the probability that two of these would be different genomic fragments labeled with identical barcodes is only 0.0031 (for details of this probability calculation, see Miner et al., Nucl. Acids Res. 32(17):e135, 2004, Supplementary Materials).

In addition to adding the random barcode, molecules were “batch-stamped” by encoding the hairpin linker with information that would designate the sample analyzed and the date of analysis. Multiple variants of the hairpin linker were designed by changing nucleotides in the stem of the linker. These stem changes represented different batches of linkers, each of which were used for the analysis of a different sample. Thus, the resulting sequences each bear a consistent “batch-stamp” encoded in the stem, and a randomly variable barcode encoded in the loop (FIG. 3).

An enhanced hairpin-bisulfite PCR method was applied to the FMR1 promoter region in the DNA of males with fragile X syndrome. The classes of sequences recovered included hypermethylated sequences with distinctive barcodes and patterns of methylation (FIG. 4A), redundant hypermethylated sequences with identical barcodes and methylation patterns (FIGS. 4B and 4C), hypomethylated sequences with distinctive barcodes (FIG. 4D), redundant hypomethylated sequences with identical barcodes (FIGS. 4E and 4F), and contaminant sequences with an original linker that predates the barcoding (FIG. 4G). The number of sequences cloned influenced the observed proportion of redundancy among the recovered sequences; the observed proportions of both redundancy and contamination appeared to depend on the initial amount of DNA used and the quality of the bisulfite conversion. Among eight different DNA samples analyzed, the proportion of sequences that were redundant ranged from 7 to 51%, and the proportion of sequences that were contaminants ranged from 0 to 14%. Occasionally, contaminant sequences were cloned from PCR reactions in which control reactions (those without template DNA) showed no DNA bands on ethidium-bromide-stained agarose gels. In these contexts, bar-coding serves as a highly accurate method for positive identification of desired sequences.

Within 142 barcodes recovered from multiple reactions with FMR], the average nucleotide composition was 54% T, 26% G and 19% A. This bias is similar to that previously reported for the influence of loop nucleotides on the stability of DNA hairpin structures (Senior et al., Proc. Natl. Acad. Sci USA 85:6242-6246, 1988).

3. Discussion

The concept of molecular bar-coding has previously been used in signature-tagged mutagenesis (Hensel et al., Science 269:400-403, 1995; Shoemaker et al., Nature Genet. 14:450-456, 1996), to track the origins of expressed sequence tags (Qiu et al., Plant Physiol. 133:475-481, 2003), and to label objects for identification and authentication (Cook and Cox, Biotechnol. Lett. 25:89-94, 2003; Cox, Analyst 126:545-547, 2001). Here, a similar concept was applied to the labeling of individual genomic fragments with distinct sequence tags. The ability to bar-code and “batchstamp” genomic DNA sequences from individual alleles is useful in situations where the amount of template DNA is limited, thus identifying contaminants and redundant sequences arising from template re-cloning. Contaminant sequences were identified even when multiple control (no DNA) PCR samples were negative. Bar-coding allows for quantification of the relative abundance of genomic methylation patterns or polymorphic sequences by correcting for skewing that can arise from PCR amplification or the cloning of the products. The barcoding method thus provides a definitive solution to the problem identified previously (Taylor et al., Pathology 29:309-312, 1997; Millar et al., Methods 27:108-113, 2002), in which multiple amplified sequences are derived from a single original molecule when template DNA is limited in amount or of poor quality. The method also allows for the analysis of mutations arising during PCR amplification.

While the preferred embodiment of the invention has been illustrated and described, it will be appreciated that various changes can be made therein without departing from the spirit and scope of the invention.

1. A method for bar-coding a nucleic acid molecule comprising the steps of:
(a) contacting a target nucleic acid molecule in a sample with a bar-coded oligonucleotide under suitable conditions to anneal the bar-coded oligonucleotide to the target nucleic acid molecule, wherein the bar-coded oligonucleotide comprises a first sequence complementary to the target nucleic acid molecule, a second sequence providing a random barcode, and a third sequence that is not complementary to any sequence in the sample; and
(b) extending the annealed bar-coded oligonucleotide using the target nucleic acid molecule as a template to produce a bar-coded target nucleic acid molecule.

2. The method of claim 1, wherein the 5′ end of the bar-coded oligonucleotide comprises a tethering sequence that is complementary to a sequence 5′ to the first sequence.

3. The method of claim 1, wherein the bar-coded oligonucleotide further comprises a fourth sequence providing experimental identification information.

4. The method of claim 1, wherein the target nucleic acid molecule is a DNA molecule.

5. A method for authenticating a DNA amplification product comprising the steps of:
(a) contacting a target nucleic acid molecule in a sample with a bar-coded oligonucleotide under suitable conditions to anneal the bar-coded oligonucleotide to the target nucleic acid molecule, wherein the bar-coded oligonucleotide comprises a first sequence complementary to the target nucleic acid molecule, a second sequence providing a random barcode, and a third sequence that is not complementary to any sequence in the sample, and wherein the 5′ end of the bar-coded oligonucleotide comprises a tethering sequence that is complementary to a sequence 5′ to the first sequence;
(b) extending the annealed bar-coded oligonucleotide using the target nucleic acid molecule as a template to produce a bar-coded target nucleic acid molecule;
(c) amplifying the bar-coded target nucleic acid molecule using a primer that binds to the third sequence to produce an amplification product; and
(d) authenticating the amplification product by detecting the presence of the second sequence in the amplification product.

6. The method of claim 5, wherein the bar-coded oligonucleotide further comprises a fourth sequence providing experimental identification information and wherein step (d) further comprises authenticating the amplification product by detecting the presence of the fourth sequence in the amplification product.

7. The method of claim 5, wherein the target nucleic acid molecule is a DNA molecule.

8. A method for authenticating a DNA amplification product comprising the steps of:
(a) ligating a hairpin linker to a double-stranded target DNA molecule to produce a ligated target DNA molecule, wherein the hairpin linker comprises a first sequence providing experimental identification information, a second sequence providing a random barcode comprising nucleotides selected from the group consisting of adenosines, guanidines, and thymidines, and a third sequence complementary to the first sequence;
(b) treating the ligated target DNA molecule of step (a) under suitable conditions to convert cytosines in the ligated target DNA molecule to uracils;
(c) amplifying the treated ligated target DNA molecule of step (b) to produce an amplification product; and
(d) authenticating the amplification product of step (c) by detecting the presence of the first and second sequences in the amplification product.

9. A composition comprising a target nucleic acid molecule and a bar-coded oligonucleotide, wherein the bar-coded oligonucleotide comprises a first sequence complementary to the target nucleic acid molecule, a second sequence providing a random barcode, and a third sequence that is not complementary to any sequence in the sample, and wherein the 5′ end of the bar-coded oligonucleotide comprises a tethering sequence that is complementary to a sequence 5′ to the first sequence.

10. The composition of claim 9, wherein fourth sequence provides experimental identification information.
